(19) United States
(12) Patent Application Publication
(10) Pub. No.: US 2009/0070896 A1
(43) Pub. Date: Mar. 12, 2009

(54) INSECT RESISTANT TRANSGENIC TURF GRASS

(75) Inventors: Mitsugu Horita, Misawa Kitahiroshima-Shi (JP); Shin-Ichiro Asano, Sapporo (JP)

Correspondence Address: MORRISON & FOERSTER LLP, 425 MARKET STREET, SAN FRANCISCO, CA, 94105-2482, US

(73) Assignee: PHYLLOM LLC, Mountain View, CA (US)

(21) Appl. No.: 12/064,960

(22) PCT Filed: Aug. 30, 2006
(86) PCT No.: PCT/US2006/033940

§ 371 Date: Oct. 27, 2008

Related U.S. Application Data
(60) Provisional application No. 60/713,193, filed on Aug. 30, 2005.

(51) Int. Cl. C12N 15/82; A01H 5/00; C12N 15/11

(52) U.S. Cl. 800/279; 800/302; 536/23.6

(57) ABSTRACT

This invention relates to transgenic turf grass having resistance to turf grass pest insects. Methods of producing such insect-resistant transgenic turf grass lines are disclosed. The invention further relates to the use of the insect-resistant transgenic turf grass to eliminate or reduce the usage of spray-on insecticides to protect the turf grass from insect damage.

### RELATED APPLICATION

This application claims priority to U.S. Provisional Patent Application Ser. No. 60/713,193 filed Aug. 30, 2005.

### BACKGROUND

Bacillus thuringiensis (Bt) is a spore-forming, rod-shaped, gram-positive bacterium closely related to Bacillus cereus, A large number of Bt isolates have been found and grouped into subspecies, such as B. thuringiensis thuringiensis, B. thuringiensis kurstaki, B. thuringiensis aizawai, etc., based on a classification scheme originally developed by Bonnefoi and de Barjac in 1963. Bt is clearly distinguished from other bacilli by the production of intracellular crystals, which are insoluble deposits of proteins many of which have insecticidal activity against various insect species. During the sporulation process, Bt synthesizes large amounts of one or more of these proteins which then crystallize into a variety of shapes.

The gene coding for the crystal protein in Bt is called “cry” because of its crystal-producing phenotype. The first cry gene, later designated as cry1Aa, was cloned about 20 years ago by Schnepf and Whiteley (1981, Proc. Natl. Acad. Sci. USA 78, 2893-2897). Since then, numerous reports of the cloning of additional cry genes have been published.

Bt produce a variety of crystal proteins that differ in insect specificities, even within one strain. Most Bt produce crystalline insecticidal proteins active against Lepidoptera species. In addition, there are Bt isolates that produce crystalline insecticidal proteins active against Diptera and Coleoptera insect species. Among Bt crystal proteins, Cry3, Cry7, Cry8, Cry9, and Cry43 are known to be active against Coleoptera species. Of those, Cry8Ca is reported to be active against scarab beetles (Ohba et al., 1992, J. Appl. Microbiol. 14, 54-57). In 2003, Asano et al., (2003, Biological Control 28 (2003), 191-196) disclosed the finding of a new Bt strain called SDS-502 that showed a very high level of activity against Anomala cuprea, A. orientalis, and Popillia japonica, which are species of scarab beetles. P. japonica, the Japanese beetle is of particular importance in the U.S. It was accidentally introduced into the US and became a serious pest of turf and garden plant species such as ornamental plants. The larvae of this insect eat grass roots which results in serious destruction of turf grass. Adult Japanese beetles eat various crops and ornamental plants including roses and berries. The gene responsible for the high P. japonica activity of SDS-502 has been isolated and named cry8Da. The protein encoding this gene, Cry8Da, was found to possess a high specific activity against Japanese beetles, at least twice as high as the specific activity of Cry8Ca and Cry43Aa, two other Bt crystal proteins with insecticidal activity against Japanese beetles and other scarabs.

Recently, selected Bt cry genes have been expressed in crop plants for insect control. Technical and commercial success has been obtained in corn expressing the cry1Ab gene and cotton expressing the cry1Ac gene. However, neither transgenic turf grass nor ornamental plants showing insect resistance have been reported. There have been reports of transformed turf grass having anti-fungal and anti-herbicidal traits (e.g., Cho, et al., 2000, Plant Cell Reports 19, 1084-1089) but none demonstrating the transformation of insect-resistant traits in turf grass or ornamental plants. This is due to a number of unresolved obstacles. For example, a high transformation efficiency in highly competent turf grass cells is very important for introducing an insect resistant trait but serious technical difficulties has impeded progress in successfully transforming turf grass cells. Moreover, a high efficiency of regenerating the whole plant from the transformed cell is also crucial in creating a transgenic turf grass or transgenic ornamental plant resistant to the larvae and/or adults of Japanese beetles. This too has proved to have serious technical difficulties impeding progress in the ability to generate a complete transgenic turf grass plant or complete ornamental plant.

Besides the technical difficulties in producing transformable plant cells or calli, the lack of the availability of a potent insect resistant gene, especially one highly active against Scarabidae species, was considered to be another major hurdle to the creation of insect resistant transgenic turf grass and other ornamental plants. One of the Applicants of the present invention has discovered a Bt cry gene, cry8Da, that encodes a protein highly potent to scarab beetles (see U.S. Patent Application No. 20030017967, herein incorporated by reference in its entirety). The present invention has overcome the technical challenges to the creation of insect-resistant transgenic turf grass by demonstrating that an insect resistant trait can be introduced to turf grass with the subsequent transformed turf grass having the ability to grow to a whole plant having resistance to insects.

### SUMMARY

The present invention includes transgenic turf grass showing resistance to certain insects that attack turf grass. The invention also includes methods of making the insect resistant transgenic turf grass. The present invention also provides for uses of the transgenic grass to protect the turf from insect attack.

The present invention utilizes a method to produce highly competent (transformable) turf grass calli that are capable of regenerating into whole grass plants.

In one embodiment, a transgenic grass having an insect-active gene, cry8Da, found in the Bt SDS-502 strain is provided.

In another embodiment, different Bt insect resistant genes are provided to transform competent grass calli. These Bt genes confer insect resistance against different insect species. While the cry8Da gene has specificity for scarab beetles, another Bt gene, cry1Ca, confers resistance to the armyworm complex such as beet armyworm, and also finds use in the present invention.

In yet another embodiment, different markers that differentiate the transformed grass cells from non-transformed cells are provided.

In a further embodiment, a transgenic turf grass containing an insect-resistant gene. The insect-resistant gene may be obtained from Bacillus thuringiensis, for example, the cry8Da from Bacillus thuringiensis strain SDS-502.

In a further embodiment, the insect-resistant gene confers resistance to insects chosen from Southern masked chafer, Cyclocephala immaculata; Turfgrass masked chafers, Cyclocephala hirta and C. pasadenae; June or May beetle, Cotinis nitida; Rose chafer, Macrodactylus subspenosus; European chafer, Amphymallon majalis; Pale brown chafer, phyllopertha diversa; Chestnut brown chafer, Adoretus tenuimaculatus; Oriental beetle, Anomala orientalis; Japanese beetle, Popillia japonica; Soy bean beetle, Anomala rufocuprea; Cupreous chafer, Anomala cuprea; Black turfgrass Ataenius, Ataenius spretulus; Beet armyworm, Spodoptera exigua; The armyworm, Pseudaletia unipuncta; Black cutworm, Agrotis ipsilon; Variegated cutworm, Peridroma saucia; Granulate cutworm, Agrotis subterranean; Lucerne moth, Nomophila noctuella; Western lawn moth, Tehama bonifatella; and Sperry's lawn moth, Crambus speryellus.

The insect-resistant gene may be obtained from bacteria which are pathogenic to turf grass pest insects. In one embodiment, the gene may be obtained from a microorganism chosen from Bacillus thuringiensis, Bacillus popilliae (i.e., Paenibacillus lentimorbus) and Bacillus larvae strains.

The insect-resistant gene may code for insect-active proteins chosen from Cry1Aa1, Cry1Aa2, Cry1Aa3, Cry1Aa4, Cry1Aa5, Cry1Aa6, Cry1Aa7, Cry1Aa8, Cry1Aa9, Cry1Aa10, Cry1Aa11, Cry1Aa12, Cry1Aa13, Cry1Aa14, Cry1Ab1, Cry1Ab2, Cry1Ab3, Cry1Ab4, Cry1Ab5, Cry1Ab6, Cry1Ab7, Cry1Ab8, Cry1Ab9, Cry1Ab10, Cry1Ab11, Cry1Ab12, Cry1Ab13, Cry1Ab14, Cry1Ab15, Cry1Ab16, Cry1Ac1, Cry1Ac2, Cry1Ac3, Cry1Ac4, Cry1Ac5, Cry1Ac6, Cry1Ac7, Cry1Ac8, Cry1Ac9, Cry1Ac10, Cry1Ac11, Cry1Ac12, Cry1Ac13, Cry1Ac14, Cry1Ac15, Cry1Ad1, Cry1Ad2, Cry1Ae1, Cry1Af1, Cry1Ag1, Cry1Ah1, Cry1Ai1, Cry1Ba1, Cry1Ba2, Cry1Ba3, Cry1Ba4, Cry1Bb1, Cry1Bc1, Cry1Bd1, Cry1Bd2, Cry1Be1, Cry1Be2, Cry1Bf1, Cry1Bf2, Cry1Bg1, Cry1Ca1, Cry1Ca2, Cry1Ca3, Cry1Ca4, Cry1Ca5, Cry1Ca6, Cry1Ca7, Cry1Ca8, Cry1Ca9, Cry1Ca10, Cry1Cb1, Cry1Cb2, Cry1Da1, Cry1Da2, Cry1Db1, Cry1 Db2, Cry1Ea1, Cry1Ea2, Cry1Ea3, Cry1Ea4, Cry1Ea5, Cry1Ea6, Cry1Eb1, Cry1Fa1, Cry1Fa2, Cry1Fb1, Cry1Fb2, Cry1Fb3, Cry1Fb4, Cry1Fb5, Cry1Ga1, Cry1Ga2, Cry1Gb1, Cry1Gb2, Cry1Gc, Cry1Ha1, Cry1Hb1, Cry1Ia1, Cry1Ia2, Cry1Ia3, Cry1Ia4, Cry1Ia5, Cry1Ia6, Cry1Ia7, Cry1Ia8, Cry1Ia9, Cry1Ia10, Cry1Ia11, Cry1Ib1, Cry1Ic1, Cry1Ic2, Cry1Id1, Cry1Ie1, Cry1If1, Cry1Ja1, Cry1Jb1, Cry1Jc1, Cry1Jc2, Cry1Jd1, Cry1Ka1, Cry2Aa1, Cry2Aa2, Cry2Aa3, Cry2Aa4, Cry2Aa5, Cry2Aa6, Cry2Aa7, Cry2Aa8, Cry2Aa9, Cry2Aa10, Cry2Aa11, Cry2Ab1, Cry2Ab2, Cry2Ab3, Cry2Ab4, Cry2Ab5, Cry2Ab6, Cry2Ac1, Cry2Ac2, Cry2Ac3, Cry2Ad1, Cry2Ae1, Cry3Aa1, Cry3Aa2, Cry3Aa3, Cry3Aa4, Cry3Aa5, Cry3Aa6, Cry3Aa7, Cry3Ba1, Cry3Ba2, Cry3Bb1, Cry3Bb2, Cry3Bb3, Cry3Ca1, Cry4Aa1, Cry4Aa2, Cry4Aa3, Cry4Ba1, Cry4Ba2, Cry4Ba3, Cry4Ba4, Cry4Ba5, Cry5Aa1, Cry5Ab1, Cry5Ac1, Cry5Ba1, Cry6Aa1, Cry6Aa2, Cry6Ba1, Cry7Aa1, Cry7Ab1, Cry7Ab2, Cry8Aa1, Cry8Ba1, Cry8Bb1, Cry8Bc1, Cry8Ca1, Cry8Ca2, Cry8Da1, Cry8Da2, Cry8Da3, Cry8Ea1, Cry9Aa1, Cry9Aa2, Cry9Ba1, Cry9Ca1, Cry9Ca2, Cry9Da1, Cry9Da2, Cry9Ea1, Cry9Ea2, Cry9Eb1, Cry9Ec1, Cry10Aa1, Cry10Aa2, Cry10Aa3, Cry11Aa1, Cry11Aa2, Cry11Aa3, Cry11Ba1, Cry11Bb1, Cry12Aa1, Cry13Aa1, Cry14Aa1, Cry15Aa1, Cry16Aa1, Cry17Aa1, Cry18Aa1, Cry18Ba1, Cry18Ca1, Cry19Aa1, Cry19Ba1, Cry20Aa1, Cry21Aa1, Cry21Aa2, Cry21Ba1, Cry22Aa1, Cry22Aa2, Cry22Ab1, Cry22Ab2, Cry22Ba1, Cry23Aa1, Cry24Aa1, Cry25Aa1, Cry26Aa1, Cry27Aa1, Cry28Aa1, Cry28Aa2, Cry29Aa1, Cry30Aa1, Cry30Ba1, Cry31Aa1, Cry31Aa2, Cry32Aa1, Cry32Ba1, Cry32Ca1, Cry32Da1, Cry33Aa1, Cry34Aa1, Cry34Aa2, Cry34Ab1, Cry34Ac1, Cry34Ac2, Cry34Ba1, Cry35Aa1, Cry35Aa2, Cry35Ab1, Cry35Ab2, Cry35Ac1, Cry35Ba1, Cry36Aa1, Cry37Aa1, Cry38Aa1, Cry39Aa1, Cry40Aa1, Cry40Ba1, Cry41Aa1, Cry41Ab1, Cry42Aa1, Cry43Aa1, Cry43Ba1, Cry44Aa, Cry45Aa, Cry46Aa, Cry47Aa, Cyt1Aa1, Cyt1Aa2, Cyt1Aa3, Cyt1Aa4, Cyt1Aa5, Cyt1Ab1, Cyt1Ba1, Cyt2Aa1, Cyt2Aa2, Cyt2Ba1, Cyt2Ba2, Cyt2Ba3, Cyt2Ba4, Cyt2Ba5, Cyt2Ba6, Cyt2Ba7, Cyt2Ba8, Cyt2Ba9, Cyt2Bb1, Cyt2Bc1, Cyt2Ca1, Vip3A(a) and Vip3A(b) and Vip3a(b).

The insect-resistant gene may be obtained from a microorganism chosen from Serratia species, Photorhabdus species, and Xenorhabdus species. The Serratia species may include S. proteamaculans, S. entomophila, P. fluorescens, X. nematophila and X. bovienii.

The insect-resistant gene may include the cry8Ca gene from the Bacillus thuringiensis strain buibui, the cry43A gene from Bacillus popilliae or the Bacillus thuringiensis cry1Ca gene.

The present invention is further directed to a method to reduce or eliminate any chemical or biological insecticide spray on turf grass to control insect pests by introducing one or more insect-resistant genes to turf grass. The transgenic turf grass may be chosen from Alkali Sacaton (Sporobolus airoides); Altai Wildrye (Leymus angustus); Annual Ryegrass (Lolium multiflorum); Bahiagrass (Paspalum notatum); Barley (Elyhordeum); Bermudagrass (Cynodon dactylon); Bluestem (Andropogon); Bromegrass (Bromus); Broomcorn Millet (Panicum miliaceum); Browntop (Microstegium); Buckwheat (Eriogonum); Buffalograss (Buchloe dactyloides); Bulbous Canarygrass (Phalaris aquatica); California brome, Alaska brome (Bromus sitchensis), Canada Bluegrass (Poa compressa); Canarygrass (Phalaris); Chewings Fescue (Festuca rubra); Cockspur Grass (Echinochloa); Colonial Bentgrass (Agrostis tenuis); Common Barley (Hordeum vulgare); Common Wheat (Triticum aestivum); Creeping Bentgrass (Agrostis stolonifera); Creeping Meadow Foxtail (Alopecurus arundinaceus); Crested Wheatgrass (Agropyron cristatum); Dahurian Wildrye (Elymus dahuricus); Dallisgrass (Paspalum dilatatum); Fescue (Festuca); Fineleaf Sheep Fescue (Festuca filiformis); Finger Millet (Eleusine coracana); Gamagrass (Tripsacum); Grain Sorghum (Sorghum bicolor); Grama (Bouteloua); Grazing Bromegrass (Bromus stamineus); Hard Fescue (Festuca trachyphylla); Indiangrass (Sorghastrum nutans); Intermediate Wheatgrass (Thinopyrum intermedium); Japanese Millet (Echinochloa esculenta); Kentucky Bluegrass (Poa pratensis); Kikuyugrass (Pennisetum clandestinum); Klinegrass (Panicum coloratum); Lovegrass (Eragrostis); Meadow Brome (Bromus commutatus); Meadow Fescue (Festuca pratensis); Meadow Foxtail (Alopecurus pratensis); Meadow Ryegrass (Lolium pratense); Milletgrass (Milium); Oat (Avena); Orchardgrass (Dactylis glomerata); Pearlmillet (Pennisetum americanum); Perennial Ryegrass (Lolium perenne); Prairie grass (Bromus wildenowii); Prairie Junegrass (Koeleria macrantha); Rat-Tail Fescue (Vulpia myuros); Red Fescue (Festuca rubra); Redtop (Agrostis gigantea); Reed Canarygrass (Phalaris arundinacea); Rough Bluegrass (Poa trivialis); Rush Wheatgrass (Thinopyrum ponticum); Russian Wildrye (Psathyrostachys juncea); Rye (Secale cereale) Ryegrass (Lolium); Sheep Fescue (Festuca ovina); Slender Creeping Red Fescue (Festuca rubra); Smooth Bromegrass (Bromus inermis); Sorghum (Sorghum bicolor); Streambank Wheatgrass (Elymus lanceolatus); Sudangrass (Sorghum bicolor); Switchgrass (Panicum virgatum); Tall Fescue (Lolium arundinaceum); Tall Oatgrass (Arrhenatherum elatius); Tall Wheatgrass (Thinopyrum ponticum); Timothy (Phleum pratense); Triticale (Triticosecale rimpaui); Tufted Hairgrass (Deschampsia caespitosa); Western Wheatgrass (Pascopyrum smithii); Wheat (Triticum); Wheatgrass (Agropyron); and Wildrye (Elymus) species.

In a further embodiment, a method of creating insect-resistant turf grass, including: a) introducing one or more plasmids comprising one or more insect-resistant genes into one or more turf grass calli wherein said calli are transformed; b) culturing the calli; c) growing the calli into mature turf grass; and d) testing the mature turf grass for insect resistance.

### BRIEF DESCRIPTION OF THE DRAWINGS

FIG. 1 depicts PCR analysis of transformed turf grass showing the cry8Da gene in a form of a 2 kb band (right three lanes). The left lane is a size marker. The second lane from left is a negative control obtained from non-transgenic grass.

FIG. 2 depicts a picture of a pot containing several lines of transgenic turf grass. The pot contains two third instar Japanese beetle larvae, which were allowed to consume the grass roots for one month. Some plants showed resistance while the others were killed by the insects.

FIGS. 3A-3D present the sequences for SEQ ID NO:3 and SEQ ID NO:10.

### DETAILED DESCRIPTION

### A. Overview of the Invention

The present invention provides transgenic turf grass and ornamental plants that have been transformed with insect-resistant genes from various Bacillus microorganisms such as the cry8Da gene derived from the Bacillus thuringiensis (Bt) strain SDS-502. The resultant transformed turf grass and ornamental plants are resistant to insect predation. The present invention also provides methods for transforming turf grass and ornamental plants with insect-resistant genes derived from Bacillus microorganisms such as the cry8Da obtained from SDS-502.

Insecticidal crystal proteins such as cry8Da are aggregates of a large protein (about 130-140 kDa) that is actually a protoxin—it must be activated before it has any effect. The crystal protein is highly insoluble in normal conditions, so it is entirely safe to humans, higher animals and most insects. However, it is solubilized in reducing conditions of high pH (above about pH 9.5) the conditions commonly found in the midgut of lepidopteran larvae. For this reason, crystal proteins from Bt are highly specific insecticidal agents.

Once it has been solubilized in the insect gut, the protoxin is cleaved by a gut protease to produce an active toxin of about 60 kDa. This toxin is termed delta-endotoxin. It binds to the midgut epithelial cells, creating pores in the cell membranes and leading to equilibration of ions. As a result, the gut is rapidly immobilized, the epithelial cells lyse, the larva stops feeding, and the gut pH is lowered by equilibration with the blood pH.

Transgenic plants such as turf grass and ornamental plants transformed with genes encoding insecticidal proteins, such as the cry8Da gene, are protected against insect infestation and predation by expressing the crystal protein, which acts in the same manner as if the crystal protein was exogenously applied as a biopesticide to the plant itself. In this manner, insect resistance is conferred to the transgenic plant species which, in their wild-type state, are normally a favorite target of feeding by insects.

### B. General Techniques

Practice of the present invention will generally utilize, unless otherwise indicated, conventional techniques of molecular biology, microbiology, cell biology, biochemistry, and immunology, which are within the skill of the art. Such techniques are fully explained in the literature, for example, in Cell Biology: A Laboratory Notebook (J. E. Cellis, ed., 1998); Current Protocols in Molecular Biology (F. M. Ausubel et al., eds, 1987); Short Protocols in Molecular Biology (Wiley and Sons, 1999). Furthermore, procedures employing commercially available assay kits and reagents will typically be used according to manufacturer defined protocols unless otherwise noted.

### C. DEFINITIONS

An “exogenous DNA segment”, “heterologous sequence”, “heterologous nucleic acid”, or a “heterologous gene”, as used herein, is one that originates from a source different from the particular host cell or organism, or, if from the same source, is modified in polynucleotide or amino acid sequence. Therefore, a heterologous gene in a host cell or organism includes a gene that is endogenous to the particular host cell, but has come from a different source or been otherwise modified. Modification of a heterologous sequence in the invention described herein typically occurs through the use of different DNA segments linked together to produce a heterologous gene. Or a heterologous gene can be made by nucleotide synthesis. The terms refer to a DNA segment which is foreign or heterologous to the cell, or homologous to the cell, or a combination of heterologous and homologous gene sequences in a position within the host cell nucleic acid in which the element is not ordinarily found. When exogenous DNA segments are expressed they yield exogenous polypeptides.

The term “gene” is used broadly to refer to any segment of DNA associated with a biological function. Genes include coding sequences and/or the regulatory sequences required for their expression as well as sequences that allow combinatorial functions as in the case of the present invention where two genes are fused via a linker sequence to produce a single new gene with more complex biological functions. Genes also include non-expressed DNA segments that have a variety of functions needed for the expression of that gene such as recognition sequences for other proteins. Genes can be obtained from a variety of sources, including cloning from a source of interest such as any living organism, or synthesizing from known or predicted sequence information, and may include artificial sequences designed to have desired characteristics.

The term “transgenic” when used to describe a cell or multi-cell organism indicates that the cell or organism replicates a heterologous nucleic acid, or expresses a peptide or protein encoded by a heterologous nucleic acid. Transgenic cells or organisms may contain genes that are not found within the native (non-recombinant) form of the cell. Transgenic cells or organisms may also contain genes found in the native form of the cell or organism except that the genes are modified and re-introduced into the cell by artificial means. The term also encompasses cells or organisms that contain a nucleic acid endogenous to the cell that has been modified without removing the nucleic acid from the cell or organism; such modifications include those obtained by gene replacement, site-specific mutation, and related techniques.

The term “insect resistance” means a trait of a gene, cell or multi-cell organism that confers resistance to pest insect attack (e.g., insect infestation or insect predation of the cell or multi-cell organism). The trait includes the capability of killing, repelling insects, and/or inhibiting the eating by insects. In the case of insect-resistant plants, it includes the characteristics that pest insects do not feed on the plants due to one or more insect-active (e.g., insecticidal or insectistatic) compounds produced by the plants, or due to one or more insect-repellent or feeding-inhibiting compounds, which are produced by the plants.

The term “insect-active” as used herein means a trait of a gene or protein that has insecticidal (insect killing), insect repellant, and/or insect feeding inhibition activity (insectistatic). It can be very specific to certain species of insects or have a broad spectrum of activity. In the case of broad spectral insect-active genes or proteins, they can target, or be active against, a variety of insect species and may even be active against non-insect species such as arachnids, but must have the activity against certain species of insects that are the intended target to achieve the goal of the present invention, which is to prevent turf grass and ornamental plants from insect attack.

### D. Methods and Uses of the Present Invention

The present invention relates to the production and use of transgenic turf grass demonstrating resistance to pest insects.

Using several different methods, the invention has demonstrated the capability of producing a number of transgenic turf grass lines showing strong resistance to scarab beetle attack, although the invention is not so limited as the methods and uses are applicable to the control of other insect pests by the choice of suitable insect-resistant genes for transformation into turf grass.

In order to make insect-resistant transgenic turf grass, Applicants improved the efficiency of generating transformable turf grass calli and the efficiency of regenerating whole plants from the transformed calli. Applicants conducted an extensive search of different culture conditions and found several conditions that produce the competent calli with efficiencies high enough to produce several insect-resistant transgenic lines. In order to produce insect-resistant transgenic turf grass, Applicants used a condition which was found to produce highly competent grass calli that were capable of regenerating, at a reasonable rate, whole grass plant. Applicants' experiments and the fact that no transgenic grass having the insect-resistant trait has been reported have shown that induction of the highly competent (i.e., transformable) turf grass calli that are capable of regenerating to the whole plant is an important breakthrough.

In one example, two vectors were used to transform the competent turf grass. In one vector, pBI221, an insect-active gene was cloned along with the cauliflower mosaic virus (CaMV) 35S promoter and Agrobacterium turmefaciens Ti-plasmid NOS (nopaline synthase) terminator. It should be noted that many vectors other than pBI221 may be used as long as they can integrate into the host plant genome, and the insect-resistant gene cloned in the vector has a proper promoter and terminator sequences. The adequate selection of any one of these transformation vectors can be made by people with ordinary skill in the art.

Numerous promoters are available for expressing a gene in a transgenic plant. One skilled in the art can obtain available plant promoters from well known databases. For example, Plant CARE: A database of plant promoters and Cis-Acting Regulatory Elements at http://sphinx.rug.ac.be:8080/PlantCARE/ maintains the current list (Lescot, et al., 2002, Nucl. Acids Res., 30, 325-327).

The insect-resistant gene expressed in the transgenic plant may be a fusion protein. For example, a protoplast-targeting leader sequence can be added to the insect-resistant protein. Or, the insect-active protein may be fused with a chroloplast-targeting sequence in order to have the gene expressed in the chroloplast.

In one example in which two vectors including pBI221 were used, the other vector, the GFP (Green Fluorescent Protein) gene was cloned with the same 35S promoter and NOS terminator. These two vectors were mixed to coat the gold particles. The DNA coated particles were then shot to competent grass cells by a particle gun. Transformed grass cells showing green fluorescence were excised out and cultured further until they developed to the whole plants. In this example, GFP worked as a “reporter” gene. It reports by green fluorescence when the transformation takes place. Another example of a reporter gene is the gus gene. The glucuronidase (GUS) enzyme from E. coli (EC 3.2.1.31) has been well documented to provide desirable characteristics as a marker gene in transformed plants. Use of the gus gene and other reporter genes is well known to people skilled in the art.

This invention is not limited to the GFP reporter gene for selecting the transformed grass cells. In another example, a bialaphos herbicide-resistant gene (bar) was used. This method also produced an insect-resistant grass line. This is an example of using a “selection” gene. Only transformed grass cells can survive (thus selected) on a special tissue culture medium. Other herbicide-resistant genes that can be used to produce insect-resistant transgenic grass include glyphosate-resistant (e.g., 3-enoyl pyruvyl shikimate 5-phosphate synthase) gene, bromoxynil-resistant (e.g. bromoxynil nitrilase) gene, sulfonamides-resistant (e.g., dihydropteroate synthase) gene, and sulfonylurea-resistant (e.g., acetolactate synthase) gene. In addition, antibiotic-resistant genes such as kanamycin- and hygromycin-resistant genes can be used. On the other hand, metabolism-related genes can also be used for selecting the transformed grass cells. For example, the manA gene from E. coli (Miles et al., 1984, Gene 32, 41-48) encoding the enzyme phosphomannose isomerase can convert mannose-6-phosphate to fructose-6-phosphate, which can then be utilized by plant cells. When placed on a medium containing mannose as the sole sugar source, non-transformed cells are outgrown by the transformed cells. The use of these selection marker genes in plant transformation is well known to those of ordinary skill in the art.

In the present invention, the particle gun technology was used to transform the competent turf grass calli as shown in one example, infra. In addition, agrobacterium-mediated, floral dip, protoplast, and electroporation transformation methods can be used. All of these plant transformation methods are well known to those of skill in the art.

Re-generated whole plants were transplanted onto potted soil and challenged by Japanese beetle larvae. Japanese beetle larvae selectively consumed those plants which were not resistant to the insect (i.e., those plants which were not transformed with the appropriate insect-resistant gene) but did not consume those plants which were transformed with the appropriate insect-resistant gene (see, infra). This level of insect protection conferred by the transformation of the insect-resistant gene into turf grass was as good as the level of protection obtained with a chemical pesticide (fenthion) applied to a non-transformed turf grass plant.

In yet another example, the present invention demonstrated that Applicants' system could produce insect-resistant transgenic lines with two other grass species, tall fescue (Lolium arundinaceum) and Perennial Ryegrass (Lolium perenne).

Insect-resistant grass may be made with any of the following grass species. Suitable, though non-limiting, examples, listed with common names followed by Latin names, are as follows: Alkali Sacaton (Sporobolus airoides); Altai Wildrye (Leymus angustus); Annual Ryegrass (Lolium multiflorum); Bahiagrass (Paspalum notatum); Barley (Elyhordeum); Bermudagrass (Cynodon dactylon); Bluestem (Andropogon); Bromegrass (Bromus); Broomcorn Millet (Panicum miliaceum); Browntop (Microstegium); Buckwheat (Eriogonum); Buffalograss (Buchloe dactyloides); Bulbous Canarygrass (Phalaris aquatica); California brome, Alaska brome (Bromus sitchensis); Canada Bluegrass (Poa compressa); Canarygrass (Phalaris); Chewings Fescue (Festuca rubra); Cockspur Grass (Echinochloa); Colonial Bentgrass (Agrostis tenuis); Common Barley (Hordeum vulgare); Common Wheat (Triticum aestivum); Creeping Bentgrass (Agrostis stolonifera); Creeping Meadow Foxtail (Alopecurus arundinaceus); Crested Wheatgrass (Agropyron cristatum); Dahurian Wildrye (Elymus dahuricus); Dallisgrass (Paspalum dilatatum); Fescue (Festuca); Fineleaf Sheep Fescue (Festuca filiformis); Finger Millet (Eleusine coracana); Gamagrass (Tripsacum); Grain Sorghum (Sorghum bicolor); Grama (Bouteloua); Grazing Bromegrass (Bromus stamineus); Hard Fescue (Festuca trachyphylla); Indiangrass (Sorghastrum nutans); Intermediate Wheatgrass (Thinopyrum intermedium); Japanese Millet (Echinochloa esculenta); Kentucky Bluegrass (Poa pratensis); Kikuyugrass (Pennisetum clandestinum); Klinegrass (Panicum coloratum); Lovegrass (Eragrostis); Meadow Brome (Bromus commutatus); Meadow Fescue (Festuca pratensis); Meadow Foxtail (Alopecurus pratensis); Meadow Ryegrass (Lolium pratense); Milletgrass (Milium); Oat (Avena); Orchardgrass (Dactylis glomerata); Pearlmillet (Pennisetum americanum); Perennial Ryegrass (Lolium perenne); Prairie grass (Bromus wildenowii); Prairie Junegrass (Koeleria macrantha); Rat-Tail Fescue (Vulpia myuros); Red Fescue (Festuca rubra); Redtop (Agrostis gigantea); Reed Canarygrass (Phalaris arundinacea); Rough Bluegrass (Poa trivialis); Rush Wheatgrass (Thinopyrum ponticum); Russian Wildrye (Psathyrostachys juncea); Rye (Secale cereale); Ryegrass (Lolium); Sheep Fescue (Festuca ovina); Slender Creeping Red Fescue (Festuca rubra); Smooth Bromegrass (Bromus inermis); Sorghum (Sorghum bicolor); Streambank Wheatgrass (Elymus lanceolatus); Sudangrass (Sorghum bicolor); Switchgrass (Panicum virgatum); Tall Fescue (Lolium arundinaceum); Tall Oatgrass (Arrhenatherum elatius); Tall Wheatgrass (Thinopyrum ponticum); Timothy (Phleum pratense); Triticale (Triticosecale rimpaui); Tufted Hairgrass (Deschampsia caespitosa); Western Wheatgrass (Pascopyrum smithii); Wheat (Triticum); Wheatgrass (Agropyron); and Wildrye (Elymus).

Once a line of transgenic grass having the insect-resistant trait is made, the trait can be transferred to other grass lines by crossing as is well known to those of skill in the art.

In another aspect of the present invention, insect resistance in turf grass was achieved with Bt insect-active toxins as shown in one example, infra. In this example, a Bt insect-active gene known as cry8Da encoding the Cry8Da protein was used. This gene was cloned into a vector and used to transform the competent grass cells derived from tall fescue and perennial ryegrass. In order to make turf grass resistant to scarab beetles, Bt insect-active genes other than cry8Da may be used. For example, cry8Ca, cry18's, and cry43Aa can be used. These genes may be isolated from B. thuringiensis subsp. japonensis (cry8Ca) and B. popilliae, i.e., Paenibacillus lentimorbus (cry18's and cry43Aa). Another example teaches how to clone the cry43Aa gene in the transformation vector. These genes belong to the class of Bt cry genes even though some are from non Bt sources (cry18 and cry43Aa for example).

Other microbial toxins active against scarab beetles include those from other bacilli such as Bacillus larvae as well as from non bacillus bacteria. For example, Serratia spp., Such as S. proteamaculans and S. entomophila (Hurst et al., 2004, J. Bacteriol. 186, 5116-5128); Photorhabdus spp., Such as P. fluorescens; (Hurst et al., 2004, J. Bateriol. 186, 5116-5128, and Bowen et al., 1998, Science, 280, 2129-2132), and Xenorhabdus species such as X. nematophila and X. bovienii (Brillard et al., 2001, Appl. Environ. Microbiol., 67, 2515-2525) are known to produce proteins that are toxic to insects and are contemplated for use in the present invention.

The scope of Applicants' invention is not limited to scarab beetles. Beet armyworm, Spodoptera exigue, is a serious pest of turf grass. Using the methods of the present invention, insect resistance against Lepidopteran species can be introduced to turf grass. The insect-active genes may be isolated from Bt and expressed in turf grass to produce one or more insect-active proteins. For example, the following Bt insect-active proteins are contemplated for use in the present invention and include: Cry1Aa1, Cry1Aa2, Cry1Aa3, Cry1Aa4, Cry1Aa5, Cry1Aa6, Cry1Aa7, Cry1Aa8, Cry1Aa9, Cry1Aa10, Cry1Aa11, Cry1Aa12, Cry1Aa13, Cry1Aa14, Cry1Ab1, Cry1Ab2, Cry1Ab3, Cry1Ab4, Cry1Ab5, Cry1Ab6, Cry1Ab7, Cry1Ab8, Cry1Ab9, Cry1Ab10, Cry1Ab11, Cry1Ab12, Cry1Ab13, Cry1Ab14, Cry1Ab15, Cry1Ab16, Cry1Ac1, Cry1Ac2, Cry1Ac3, Cry1Ac4, Cry1Ac5, Cry1Ac6, Cry1Ac7, Cry1Ac8, Cry1Ac9, Cry1Ac10, Cry1Ac11, Cry1Ac12, Cry1Ac13, Cry1Ac14, Cry1Ac15, Cry1Ad1, Cry1Ad2, Cry1Ae1, Cry1Af1, Cry1Ag1, Cry1Ah1, Cry1Ai1, Cry1Ba1, Cry1Ba2, Cry1Ba3, Cry1Ba4, Cry1Bb1, Cry1Bc1, Cry1Bd1, Cry1Bd2, Cry1Be1, Cry1Be2, Cry1Bf1, Cry1Bf2, Cry1Bg1, Cry1Ca1, Cry1Ca2, Cry1Ca3, Cry1Ca4, Cry1Ca5, Cry1Ca6, Cry1Ca7, Cry1Ca8, Cry1Ca9, Cry1Ca10, Cry1Cb1, Cry1Cb2, Cry1Da1, Cry1Da2, Cry1Db1, Cry1Db2, Cry1Ea1, Cry1Ea2, Cry1Ea3, Cry1Ea4, Cry1Ea5, Cry1Ea6, Cry1Eb1, Cry1Fa1, Cry1Fa2, Cry1Fb1, Cry1Fb2, Cry1Fb3, Cry1Fb4, Cry1Fb5, Cry1Ga1, Cry1Ga2, Cry1Gb1, Cry1Gb2, Cry1Gc, Cry1Ha1, Cry1Hb1, Cry1Ia1, Cry1Ia2, Cry1Ia3, Cry1Ia4, Cry1Ia5, Cry1Ia6, Cry1Ia7, Cry1Ia8, Cry1Ia9, Cry1Ia10, Cry1Ia11, Cry1Ib1, Cry1Ic1, Cry1Ic2, Cry1Id1, Cry1Ie1, Cry1If1, Cry1Ja1, Cry1Jb1, Cry1Jc1, Cry1Jc2, Cry1Jd1, Cry1Ka1, Cry2Aa1, Cry2Aa2, Cry2Aa3, Cry2Aa4, Cry2Aa5, Cry2Aa6, Cry2Aa7, Cry2Aa8, Cry2Aa9, Cry2Aa10, Cry2Aa11, Cry2Ab1, Cry2Ab2, Cry2Ab3, Cry2Ab4, Cry2Ab5, Cry2Ab6, Cry2Ac1, Cry2Ac2, Cry2Ac3, Cry2Ad1, Cry2Ae1, Cry3Aa1, Cry3Aa2, Cry3Aa3, Cry3Aa4, Cry3Aa5, Cry3Aa6, Cry3Aa7, Cry3Ba1, Cry3Ba2, Cry3Bb1, Cry3Bb2, Cry3Bb3, Cry3Ca1, Cry4Aa1, Cry4Aa2, Cry4Aa3, Cry4Ba1, Cry4Ba2, Cry4Ba3, Cry4Ba4, Cry4Ba5, Cry5Aa1, Cry5Ab1, Cry5Ac1, Cry5Ba1, Cry6Aa1, Cry6Aa2, Cry6Ba1, Cry7Aa1, Cry7Ab1, Cry7Ab2, Cry8Aa1, Cry8Ba1, Cry8Bb1, Cry8Bc1, Cry8Ca1, Cry8Ca2, Cry8Da1, Cry8Da2, Cry8Da3, Cry8Ea1, Cry9Aa1, Cry9Aa2, Cry9Ba1, Cry9Ca1, Cry9Ca2, Cry9Da1, Cry9Da2, Cry9Ea1, Cry9Ea2, Cry9Eb1, Cry9Ec1, Cry10Aa1, Cry10Aa2, Cry10Aa3, Cry11Aa1, Cry11Aa2, Cry11Aa3, Cry11Ba1, Cry1Bb1, Cry12Aa1, Cry13Aa1, Cry14Aa1, Cry15Aa1, Cry16Aa1, Cry17Aa1, Cry18Aa1, Cry18Ba1, Cry18Ca1, Cry19Aa1, Cry19Ba1, Cry20Aa1, Cry21Aa1, Cry21Aa2, Cry21Ba1, Cry22Aa1, Cry22Aa2, Cry22Ab1, Cry22Ab2, Cry22Ba1, Cry23Aa1, Cry24Aa1, Cry25Aa1, Cry26Aa1, Cry27Aa1, Cry28Aa1, Cry28Aa2, Cry29Aa1, Cry30Aa1, Cry30Ba1, Cry31Aa1, Cry31Aa2, Cry32Aa1, Cry32Ba1, Cry32Ca1, Cry32Da1, Cry33Aa1, Cry34Aa1, Cry34Aa2, Cry34Ab1, Cry34Ac1, Cry34Ac2, Cry34Ba1, Cry35Aa1, Cry35Aa2, Cry35Ab1, Cry35Ab2, Cry35Ac1, Cry35Ba1, Cry36Aa1, Cry37Aa1, Cry38Aa1, Cry39Aa1, Cry40Aa1, Cry40Ba1, Cry41Aa1, Cry41Ab1, Cry42Aa1, Cry43Aa1, Cry43Ba1, Cry44Aa, Cry45Aa, Cry46Aa, Cry47Aa, Cyt1Aa1, Cyt1Aa2, Cyt1Aa3, Cyt1Aa4, Cyt1Aa5, Cyt1Ab1, Cyt1Ba1, Cyt2Aa1, Cyt2Aa2, Cyt2Ba1, Cyt2Ba2, Cyt2Ba3, Cyt2Ba4, Cyt2Ba5, Cyt2Ba6, Cyt2Ba7, Cyt2Ba8, Cyt2Ba9, Cyt2Bb1, Cyt2Bc1, Cyt2Ca1, Vip3A(a) and Vip3A(b) and Vip3a(b). One would generate the above-listed proteins in turf grass or ornamental plant species by using the methods of the present invention to express said proteins in plant cells (e.g., by cloning in the genes coding for the above proteins into a suitable vector and transforming plant cells as is more particularly described supra and infra).

In yet another aspect, the present invention provides a method for protecting turf grass from insect attack. According to one embodiment, beetle larvae which are commonly called “grubs” can be controlled by use of the present invention. The grubs are the larval stages of beetles such as southern masked chafer, Cyclocephala immaculata; turfgrass masked chafers, Cyclocephala hirta, C. pasadenae; June or May beetle, Cotinis nitida; rose chafer, Macrodactylus subspenosus; European chafer, Amphymallon majalis; Pale brown chafer, phyllopertha diversa; chestnut brown chafer, Adoretus tenuimaculatus; oriental beetle, Anomala orientalis; Japanese beetle, Popillia japonica; soy bean beetle, Anomala rufocuprea; cupreous chafer, Anomala cuprea and black turfgrass Ataenius, Ataenius spretulus.

In addition to these scarab beetles, the present invention can produce a transgenic turf grass line that is active against larvae of moths and butterflies. For example, resistance to cutworms and armyworms such as beet armyworm, Spodoptera exigua; the armyworm, Pseudaletia unipuncta; black cutworm, Agrotis ipsilon; variegated cutworm, Peridroma saucia; and granulate cutworm, Agrotis subterranean as well as resistance to lucerne moth, Nomophila noctuella; western lawn moth, Tehama bonifatella; and sperry's lawn moth, Crambus speryellus can be obtained.

In yet another aspect, insect-resistant ornamental plants are provided. Table 1 illustrates several ornamental plants having use in the present invention.

TABLE 1
Landscape Plants Susceptible to Attack by Adult Japanese Beetles

| Scientific name | Common name |
|---|---|
| Acer palmatum | Japanese Maple |
| Acer plananoides | Norway Maple |
| Aesculus hippocastanum | Horse chestnut |
| Betula populifolia | Gray birch |
| Castanea dentata | American chestnut |
| Nibiscus syriacus | Rose-of-Sharon, Shrub Althea |
| Juglans nigra | Black walnut |
| Malus species | Flowering crabapple, apple |
| Platanus acerifolia | London planetree |
| Populus nigra italica | Lombardy poplar |
| Prunus species | Cherry, black cherry, plum, peach |
| Rosa species | Roses |
| Sassafras albidum | Sassafras |
| Sorbus americana | American mountain-ash |
| Tilia americana | American linden |
| Ulmus americana | American elm |
| Ulmus procera | English elm |
| Vitis species | Table Grapes |

Plants which grow rapidly and are especially attractive to the beetles are most difficult to protect. Roses unfold quickly and are especially attractive to beetles. When beetles are abundant, nip buds and spray to protect the leaves or cover the roses with netting to keep beetles out.

Beetles are fond of certain weeds and non economic plants such as bracken, elder, multiflora rose, Indian mallow, sassafras, poison ivy, smartweed, wild fox grape and wild summer grape. Elimination of these plants whenever practical destroys these continuous sources of infestation.

The above-listed plants (Table 1, infra) and others are transformed with insect-resistant genes using standard molecular biological and botanical methods known to those of skill in the art, allowed to grow, and challenged by insects to determine insect resistance, as is exemplified in the Examples with transgenic turf grass, infra.

The following examples are provided to show that the present invention may be used to generate transgenic turf grass that is resistant to insects. Those skilled in the art will recognize that while specific embodiments have been illustrated and described, they are not intended to limit the invention.

### EXAMPLES

### Example 1

### Cloning the Cry8Da Gene

The cry8Da gene was cloned from Bt SDS-502 following the method described in the paper published by Asano et al. ((2003), Biological Control 28, 191-196, herein incorporated by reference in its entirety). A fragment of the cry8Da gene containing the active region was amplified by PCR using two primers having the sequences,

| | |
|---|---|
| 5′-GGATCCCATGAGTCCAAATAATCAAAATG | (SEQ ID NO: 1) |
| 5′-CCCGGGTCACACATCTAGGTCTTCTTCTGC | (SEQ ID NO: 2) |

and the cloned cry8Da gene as the template. PCR was carried out in a 100 ul reaction mixture containing 10 pg template DNA and other components at their proper concentrations (well known to those of skill in the art). In one example, the PCR mixture contained: 10 ul 10× buffer, 2 ul d-NTP, 2.5 ul Primer 1 (20 uM), 2.5 ul Primer 2 (20 uM), 2 ul Taq Polymerase, 1 ul template DNA and 80 ul water. The temperature cycling in the PCR was 96° C. (30 sec.) 45° C. (45 sec.) 72° C. (1 min. 30 sec.), for 25 cycles. The PCR amplified gene fragment was then cloned in pGEM-T-Easy (Promega, Madison, Wis., USA) following the instruction given by the plasmid manufacturer. The cloned gene was sequenced to confirm the sequence of the cry8Da gene as published in U.S. Patent Application No. 20030017967 (herein incorporated by reference in its entirety). The PCR amplified cry8Da gene in PGEM-T-Easy was excised out with BamHI and SacI utilizing the sites provided in pGEM-T-Easy and cloned in pBI221, which had been cut with BamHI and SacI to remove the gusA gene. The resultant plasmid derived from pBI221 in which the cry8Da gene was cloned was called pBI221-8D1 (SEQ ID NO:3) and used in plant transformation. Plasmids pBI221-8D1 and the other plasmid used in the plant transformation, p35S-GFP, were purchased from Clonetech (Mountain View, Calif., USA).

### Example 2

### Cloning of Beetle Active Genes Other than Cry8Da

The cry43Aa gene (GenBank Accession Number: AB115422) encodes a protein toxic to scarab beetles such as Anomala cuprea and Popillia japonica. These are serious pests of turf grass causing considerable damage by their feeding behavior. The cry43Aa gene is cloned from the Bacillus popilliae Hime strain, a strain that was isolated from a diseased cupreous chafer, Anomala cuprea. The full-length cry43Aa gene was amplified by PCR using two primers having the sequences,

| | |
|---|---|
| 5′-GGATCCATGAATCAGTATCATAACCAAAACG | (SEQ ID NO: 4) |
| 5′-CCCGGGTTACTTTTCCATACAAATCAATTCCAC | (SEQ ID NO: 5) |

In the PCR reaction mixture, 1 ul of genomic DNA prepared from the B. polilliae Hime strain containing about 100 ng of DNA was used.

The cry8Ca gene (GenBank Accession number: U04366) also encodes a protein toxic to scarab beetles. The cry8Ca gene is cloned from the Bacillus thrungiensis buibui strain, a strain which was obtained from the Fermentation Research Institute, Agency of Industrial Science and Technology, Ministry of International Trade and Industry, Japan. The full-length cry8Ca gene was amplified by PCR using two primers having the sequences,

| | |
|---|---|
| 5′-GGATCCATGAGTCCAAATAATCAAAATGAG | (SEQ ID NO: 6) |
| 5′-CCCGGGTTACTCTTCTTCTAACACGAGTTCTAC | (SEQ ID NO: 7) |

In the PCR reaction mixture, 1 ul of genomic DNA prepared from the B. thrungiensis buibui strain containing about 100 ng of DNA was used.

The PCR amplified cry43Aa and cry8Ca genes are cloned in pBI221 as described in Example I and used in turf grass transformation and follow-on experiments testing for anti-beetle activity as is described in Example 10, infra.

### Example 3

### Cloning of the Cry1Ca Gene

The cry1Ca gene (GenBank Accession number: X07518) encodes a protein toxic to the armyworm complex, such as Spodoptera exigua, S. frigiperda, Pseudaletia unipuncta, etc. These are serious pests of turf grass causing considerable damage. The cry1Ca gene was cloned from the B. thringiensis subsp. aizawai HD133 strain which was obtained from USDA, ARS, Northern Regional Research Center, Peoria, Ill., USA. The full-length cry1Ca gene was amplified by PCR using two primers having the sequences,

(SEQ ID NO: 8)

5′-GGATCCCATGGAGGAAAATAATCAAAATCAATGC

(SEQ ID NO: 9)

5′-CCCGGGTTATTCCTCCATAAGGAGTAATTCC

In the PCR reaction mixture, 1 ul of genomic DNA prepared from the HD-133 strain containing about 100 ng of DNA was used. The PCR amplified cry1Ca gene is cloned in pBI221 as described in Example 1 and used in the turf grass transformation and follow-on experiments testing for anti-beetle activity as is described in Example 10, infra.

### Example 4

### Synthesis of GC-Rich cry8Da

A synthetic cry8Da gene (SEQ ID NO:10) was obtained from Phyllom LLC, Menlo Park, Calif., USA. In this gene, the ratio between GC and AT contents was modified to increase the GC content from the naturally occurring Bt cry8Da sequence. Bt cry genes utilize codons rich in AT more often than eukaryotic organisms such as animals and plants. In the synthetic cry8Da, no changes to the peptide sequence were made. The synthesized gene has BamHI and NotI sites that allow direct cloning into pBI221 that has been cut with these restriction enzymes. The gene was cloned in pBI221 and used to transform turf grass and follow-on experiments testing for anti-beetle activity as is described in Example 10, infra.

### Example 5

### Callus Induction

Two turf grass lines, perennial ryegrass and tall fescue, were used in this example. Grass seeds were soaked in 1% sodium hypochlorite for 15 min to sterilize the seed surface. From the sterile seeds; seed coat was removed and the coat-removed seeds were soaked in sterile water overnight. The seeds were treated in 1% sodium hypochlorite for 15 min again. After the second sodium hypochlorite treatment, the seeds were dissected and the seed embryo was isolated. The embryo was then cultured on a callus-induction medium based on the MS medium (Murashige and Skoog, 1962, Physiol. Plant, 15, 473-497). In this callus-induction medium, ammonium nitrate concentration was reduced to 50% from the original MS medium recipe and 3% sucrose was added. During the experiment, various plant growth regulators were tested for the best result as shown in Table 2. Table 2 indicated the number of calli that were induced from 30 tall fescue embryo samples and the number of calli that were capable of regenerating the whole plant.

TABLE 2
Turf grass callus induction and regeneration

| PGR | CuSO₄ | Calli induced (%) | Calli Regenerated |
|---|---|---|---|
| D2 | − | 27 (90) | 6 |
| D2 | + | 19 (63) | 1 |
| D2/B001 | − | 21 (70) | 6 |
| D2/B001 | + | 24 (80) | 8 * |
| D2/B01 | − | 21 (70) | 3 |
| D2/B01 | + | 19 (63) | 1 |
| D2/T001 | − | 21 (70) | 5 |
| D2/T001 | + | 17 (57) | 3 |
| D2/T01 | − | 15 (60) | 4 |
| D2/T01 | + | 15 (60) | 3 |
| DC2 | − | 25 (83) | 1 |
| DC2 | + | 21 (70) | 3 |
| DC2/B001 | − | 15 (50) | 3 |
| DC2/B001 | + | 23 (77) | 3 |
| DC2/B01 | − | 17 (57) | 2 |
| DC2/B01 | + | 18 (60) | 3 |
| DC2/T001 | − | 17 (57) | 1 |
| DC2/T001 | + | 12 (40) | 3 |
| DC2/T01 | − | 16 (53) | 1 |
| DC2/T01 | + | 17 (57) | 2 |

PGR keys:
Auxins
D2: 2 mg/l 2,4-Dichlorophynoxyacetic acid (2,4-D)
DC2: 2 mg/l Dicamba (3,6-dichloro-2-methoxybenzoic acid)
Cytokines
B01/B001: 0.01 or 0.001 mg/l benzylamino purine (BAP)
T01/T001: 0.01 or 0.001 mg/l TDZ (thidiazuron)
CuSO₄+ indicates increase of CuSO₄ concentration by 50% from the original MS medium. CuSO₄− indicates no increase.
* this combination was used to induce calli which were used in the transformation.

From this experiment, Applicants used, in the MS medium, 2,4-Dichlorophynoxyacetic acid (2,4-D) and benzylamino purine (BAP) at 2 mg/l and 0.001 mg/l, respectively. The concentration of cupric sulfate was increased by 50%. The medium also contained 0.25% Gelrite® (Merck, North Wales, Pa., USA). Embryos on the callus-induction medium were incubated at 24° C. under 16 hr light per day for about 2 weeks until callus was formed. Every 3 weeks, callus was transferred to fresh medium and maintained until transformation.

### Example 6

### Transformation

Transformation was performed using particle gun transformation technology. Protocols contained in the user manual provided by the particle gun (GIE-III IDER) manufacturer Frontier Science, Hokkaido, Japan were followed. Calli grown on the callus-induction medium was soaked in a high osmotic pressure (HOP)-medium consisting of the entire ingredients in the callus induction-medium and 0.5 M mannitol overnight. The calli soaked in the HOP medium were cut in small sizes of about 1 mm³. About 40 callus pieces were placed on the callus-induction medium used in one transformation. Various amounts (10 to 40 ug of gold particles (1.5-3.0 micron) in 4 ul ethanol were coated with various amounts (1 ug to 4 ug) of pBI221-8D1 and p35S-GFP and shot once or twice onto callus pieces from 12 cm above the sample stage. Using GFP as an indicator of transformation efficiency, it was found that 100 ug gold particle coated with 2 ug of DNA produced the highest transformation efficiency. No significant difference in transformation efficiency was found between one shot and two shots. Therefore, a number of large scale trials were conducted under this condition. As many as 1,600 callus pieces from turf grass seeds were used in one transformation.

### Example 7

### Regeneration

The transformed callus was then transferred on to fresh medium. Each callus piece was placed on the medium about 1 cm apart from another piece. Within a few days, transformed cells showed GFP fluorescence. In one week, cell mass showing strong GFP fluorescence was excised out from each lump of callus and trans-planted on a regeneration medium. The regeneration medium is the same medium as the callus-induction medium except that no hormones were added. The transformed cells were grown on the medium at 24° C. under 16 hr light per day. Every 2 weeks, the cells were transferred to fresh medium. In 4 weeks, 3 GFP-positive tall fescue callus pieces developed into whole plants with leaves and roots.

### Example 8

### Alternative Selection Methods of Transformed Calli

Besides GFP, Bialaphos resistant (bar) gene was used as a selection marker for the transformed cells. Bialaphos is a glutamine synthetase inhibitor, and the enzyme coded by the bar gene, phosphinochricin acyltransferase, inactivates the Bialaphos. The bar gene was obtained from PGTV-BAR (Becker, et al., 1992, Plant Mol. Biol. 20, 1195-1197) along with the promoter and terminator by PCR using primers,

(SEQ ID NO: 11)

5′-CCGGAATTCGATCATGAGCGGAGAATTAAGG

(SEQ ID NO: 12)

5′-CCGGAATTCATCTTGAAAGAAATATAGTTTAAAT

and cloned in pBluescriptII-SK+ from Stratagene and used to transform the grass callus along with the pBI221-8Da1 plasmid. Some of those transformed grass cells selected by Bialaphos developed whole plants that were resistant to Bialaphos. Insect challenge as described above showed some plants were indeed resistant to Japanese beetle larvae. In another experiment, HygromycineB selection was used to select the transformed grass cells. In this case the hybromycineB resistant gene was cloned with the NOS promoter and terminator.

### Example 9

### Analysis of Transformed Grass Plants

When transformed callus developed into whole plants, a portion of leaves was taken from each plant and DNA was extracted from the leaf samples using a Qiagen DNeasy Plant Mini Kit following the instructions contained in the kit (Qiagen, Valencia, Calif., USA). The cry8Da gene in the samples were analyzed by PCR using two primers having the sequences,

| | |
|---|---|
| 5′-GGATCCCATGAGTCGAAATAATCAAAATG | (SEQ ID NO: 13) |
| 5′-CCCGGGTCACACATCTAGGTCTTCTTCTGC | (SEQ ID NO: 14) |

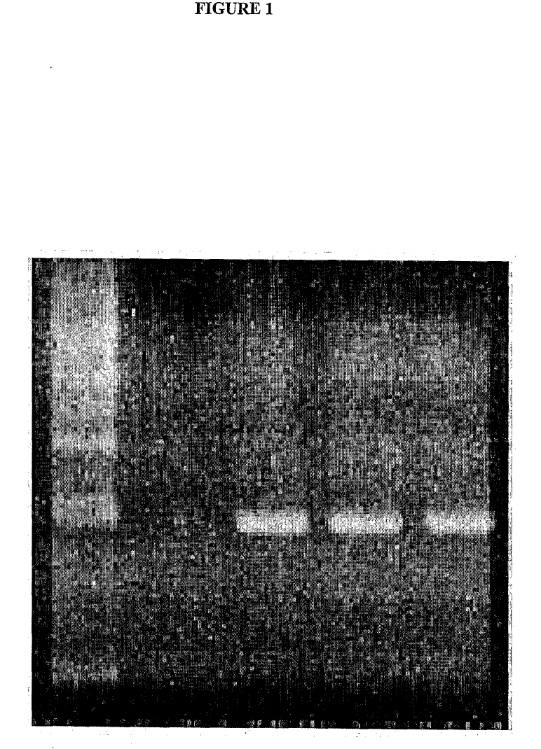

If the cry8Da gene exists in a template DNA sample (plant leaf extract), these primers should produce a 2 kb amplified fragment. All leaf samples derived from GFP-positive callus pieces showed this 2-kb fragment by PCR analysis confirming the cry8Da gene inserted into the plant genome (FIG. 1). Four lines, three from tall fescue and one from perennial ryegrass were selected based on this PCR analysis for insect resistance tests.

### Example 10

### Insect Resistance Test

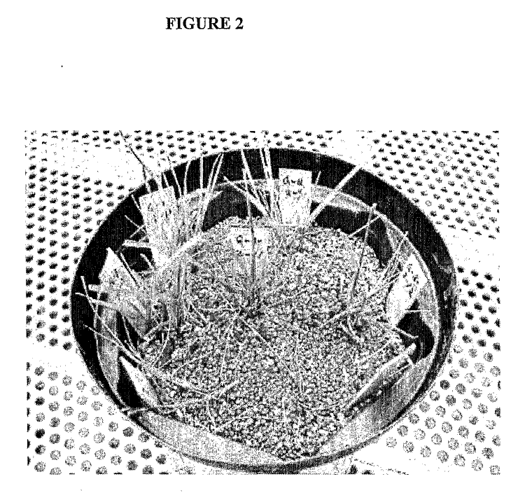

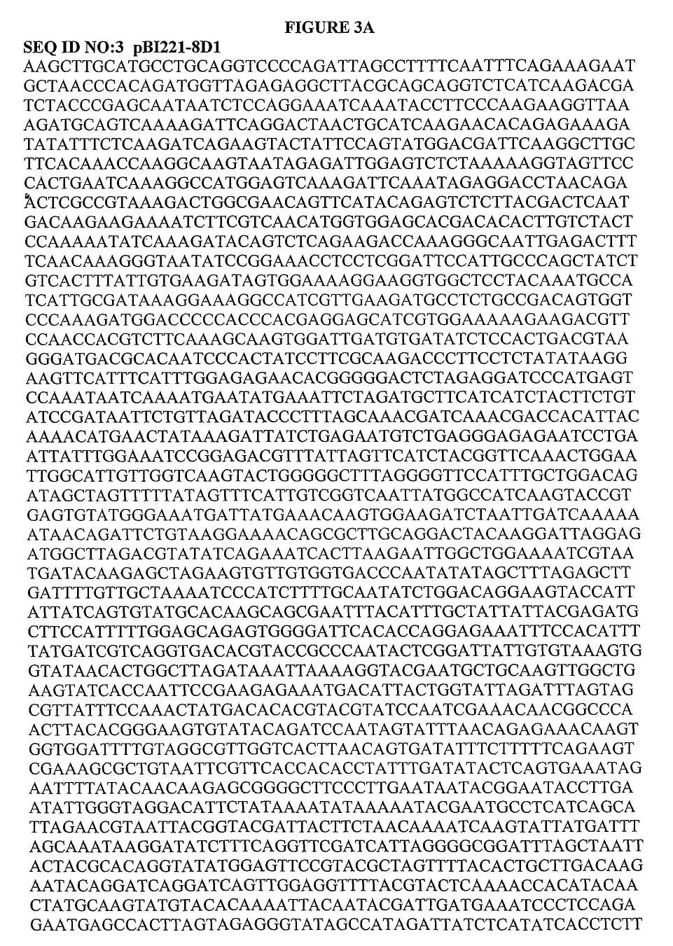

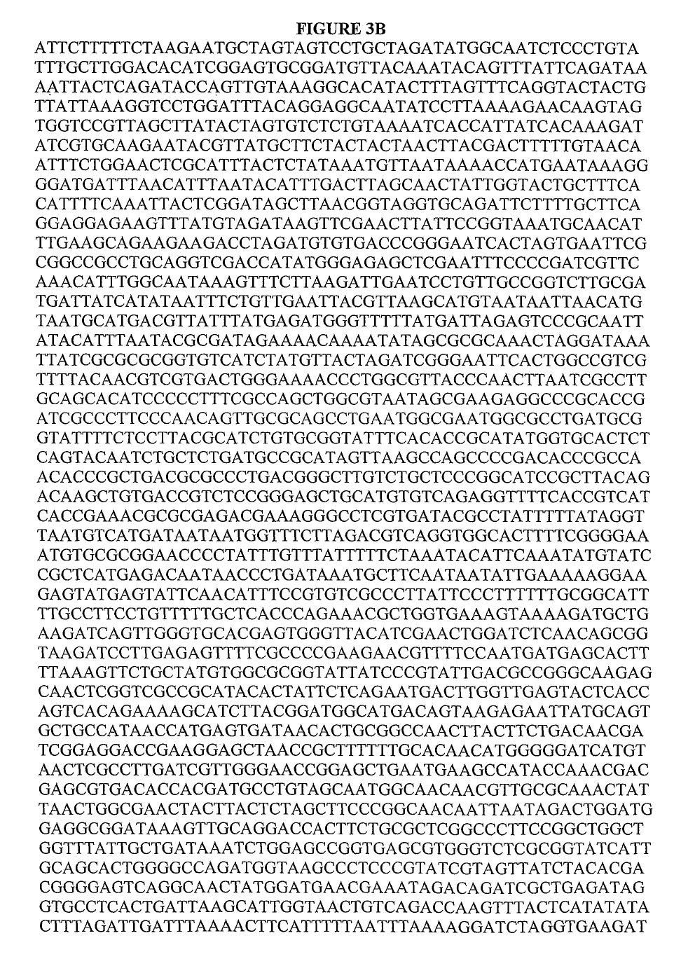

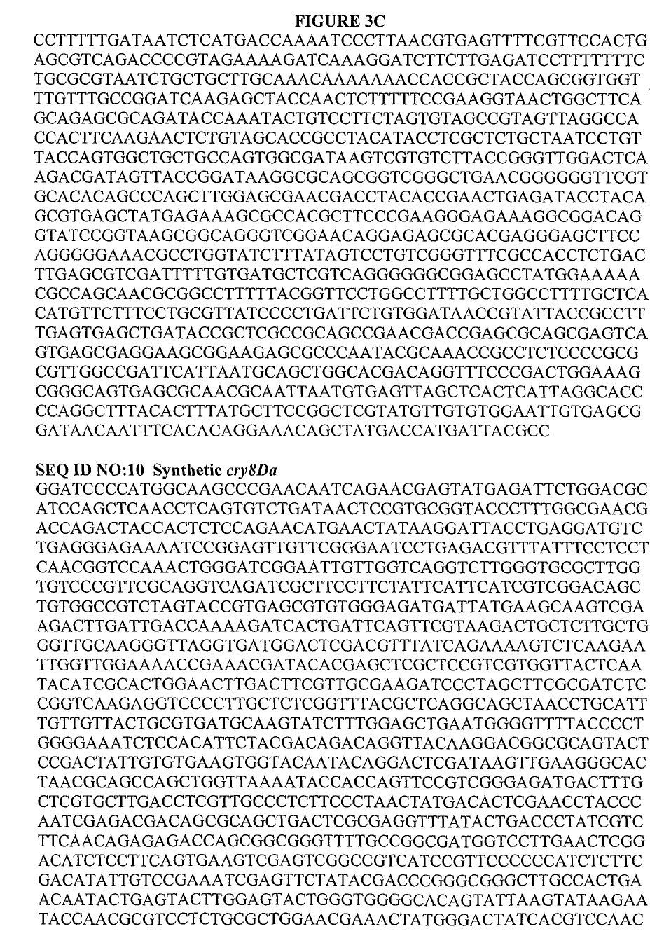

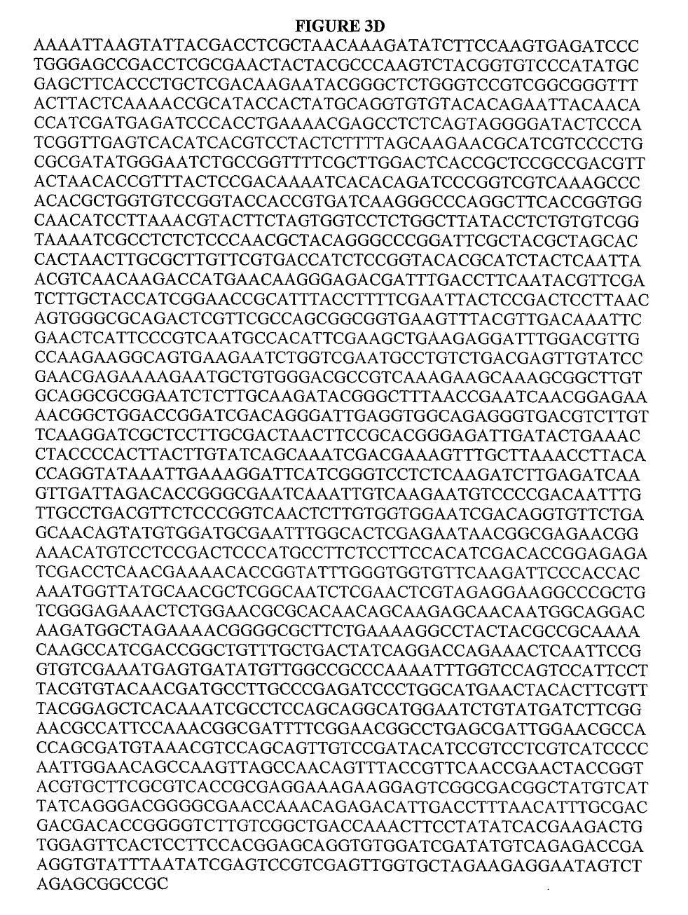

Regenerated whole plants from transformed calli were transferred to 15 cm-diameter pots containing potting soil. About 6 plants were planted in each pot. In each pot, 2 third-instar Japanese beetle larvae which had been collected from a grass field were released and were allowed to feed on grass roots for one month. The insects consumed the roots of the plants that were not resistant to the insect (i.e., were not transformed with insect-resistant genes) thereby killing the plants. However, those plants which were positive for cry8Da by PCR analysis (Example 9) showed resistance to the Japanese beetle and survived (FIG. 2). The level of protection was shown to be similar to, if not better than, the protection obtained by use of a chemical insecticide. In this example, a fenthion organophosphate insecticide was used at a rate of 0.45 Al (Active Ingredient) g/cm².

Further tests were conducted with higher insect pressure, one plant that survived through the first insect resistance test and 5 third-instar Japanese beetle larvae per pot. The cry8Da-containing grass lines also survived from this second insect resistance test with higher insect pressure. After one month of testing, all 5 insects survived in pots containing non-transformed grass while none survived in the majority of pots with transgenic grass.

1: A transgenic turf grass comprising an insect-resistant gene.

2: The transgenic turf grass of claim 1, wherein said insect-resistant gene is obtained from Bacillus thuringiensis.

3: The transgenic turf grass of claim 1, wherein said insect-resistant gene comprises cry8Da from Bacillus thuringiensis strain SDS-502.

4: The transgenic turf grass of claim 1, wherein said insect-resistant gene confers resistance to insects chosen from Southern masked chafer, Cyclocephala immaculata; Turfgrass masked chafers, Cyclocephala hirta and C. pasadenae; June or May beetle, Cotinis nitida; Rose chafer, Macrodactylus subspenosus; European chafer, Amphymallon majalis; Pale brown chafer, phyllopertha diversa; Chestnut brown chafer, Adoretus tenuimaculatus; Oriental beetle, Anomala orientalis; Japanese beetle, Popillia japonica; Soy bean beetle, Anomala rufocuprea; Cupreous chafer, Anomala cuprea; Black turfgrass Ataenius, Ataenius spretulus; Beet armyworm, Spodoptera exigua; The armyworm, Pseudaletia unipuncta; Black cutworm, Agrotis ipsilon; Variegated cutworm, Peridroma saucia; Granulate cutworm, Agrotis subterranean; Lucerne moth, Nomophila noctuella; Western lawn moth, Tehama bonifatella; and Sperry's lawn moth, Crambus speryellus.

5: The insect-resistant gene of claim 1, wherein said gene is obtained from bacteria which are pathogenic to turf grass pest insects.

6: The insect-resistant gene of claim 3, wherein said gene is obtained from a microorganism chosen from Bacillus thuringiensis, Bacillus popilliae (i.e., Paenibacillus lentimorbus) and Bacillus larvae strains.

7: The insect-resistant gene of claim 1, wherein said gene codes for insect-active proteins chosen from Cry1Aa1, Cry1Aa2, Cry1Aa3, Cry1Aa4, Cry1Aa5, Cry1Aa6, Cry1Aa7, Cry1Aa8, Cry1Aa9, Cry1Aa10, Cry1Aa11, Cry1Aa12, Cry1Aa13, Cry1Aa14, Cry1Ab1, Cry1Ab2, Cry1Ab3, Cry1Ab4, Cry1Ab5, Cry1Ab6, Cry1Ab7, Cry1Ab8, Cry1Ab9, Cry1Ab10, Cry1Ab11, Cry1Ab12, Cry1Ab13, Cry1Ab14, Cry1Ab15, Cry1Ab16, Cry1Ac1, Cry1Ac2, Cry1Ac3, Cry1Ac4, Cry1Ac5, Cry1Ac6, Cry1Ac7, Cry1Ac8, Cry1Ac9, Cry1Ac10, Cry1Ac11, Cry1Ac12, Cry1Ac13, Cry1Ac14, Cry1Ac15, Cry1Ad1, Cry1Ad2, Cry1Ae1, Cry1Af1, Cry1Ag1, Cry1Ah1, Cry1Ai1, Cry1Ba1, Cry1Ba2, Cry1Ba3, Cry1Ba4, Cry1Bb1, Cry1Bc1, Cry1Bd1, Cry1Bd2, Cry1Be1, Cry1Be2, Cry1Bf1, Cry1Bf2, Cry1Bg1, Cry1Ca1, Cry1Ca2, Cry1Ca3, Cry1Ca4, Cry1Ca5, Cry1Ca6, Cry1Ca7, Cry1Ca8, Cry1Ca9, Cry1Ca10, Cry1Cb1, Cry1Cb2, Cry1Da1, Cry1Da2, Cry1Db1, Cry1Db2, Cry1Ea1, Cry1Ea2, Cry1Ea3, Cry1Ea4, Cry1Ea5, Cry1Ea6, Cry1Eb1, Cry1Fa1, Cry1Fa2, Cry1Fb1, Cry1Fb2, Cry1Fb3, Cry1Fb4, Cry1Fb5, Cry1Ga1, Cry1Ga2, Cry1Gb1, Cry1Gb2, Cry1Gc, Cry1Ha1, Cry1Hb1, Cry1Ia1, Cry1Ia2, Cry1Ia3, Cry1Ia4, Cry1Ia5, Cry1Ia6, Cry1Ia7, Cry1Ia8, Cry1Ia9, Cry1Ia10, Cry1Ia11, Cry1Ib1, Cry1Ic1, Cry1Ic2, Cry1Id1, Cry1Ie1, Cry1If1, Cry1Ja1, Cry1Jb1, Cry1Jc1, Cry1Jc2, Cry1Jd1, Cry1Ka1, Cry2Aa1, Cry2Aa2, Cry2Aa3, Cry2Aa4, Cry2Aa5, Cry2Aa6, Cry2Aa7, Cry2Aa8, Cry2Aa9, Cry2Aa10, Cry2Aa11, Cry2Ab1, Cry2Ab2, Cry2Ab3, Cry2Ab4, Cry2Ab5, Cry2Ab6, Cry2Ac1, Cry2Ac2, Cry2Ac3, Cry2Ad1, Cry2Ae1, Cry3Aa1, Cry3Aa2, Cry3Aa3, Cry3Aa4, Cry3Aa5, Cry3Aa6, Cry3Aa7, Cry3Ba1, Cry3Ba2, Cry3Bb1, Cry3Bb2, Cry3Bb3, Cry3Ca1, Cry4Aa1, Cry4Aa2, Cry4Aa3, Cry4Ba1, Cry4Ba2, Cry4Ba3, Cry4Ba4, Cry4Ba5, Cry5Aa1, Cry5Ab1, Cry5Ac1, Cry5Ba1, Cry6Aa1, Cry6Aa2, Cry6Ba1, Cry7Aa1, Cry7Ab1, Cry7Ab2, Cry8Aa1, Cry8Ba1, Cry8Bb1, Cry8Bc1, Cry8Ca1, Cry8Ca2, Cry8Da1, Cry8Da2, Cry8Da3, Cry8Ea1, Cry9Aa1, Cry9Aa2, Cry9Ba1, Cry9Ca1, Cry9Ca2, Cry9Da1, Cry9Da2, Cry9Ea1, Cry9Ea2, Cry9Eb1, Cry9Ec1, Cry10Aa1, Cry10Aa2, Cry10Aa3, Cry11Aa1, Cry11Aa2, Cry11Aa3, Cry11Ba1, Cry11Bb1, Cry12Aa1, Cry13Aa1, Cry14Aa1, Cry15Aa1, Cry16Aa1, Cry17Aa1, Cry18Aa1, Cry18Ba1, Cry18Ca1, Cry19Aa1, Cry19Ba1, Cry20Aa1, Cry21Aa1, Cry21Aa2, Cry21Ba1, Cry22Aa1, Cry22Aa2, Cry22Ab1, Cry22Ab2, Cry22Ba1, Cry23Aa1, Cry24Aa1, Cry25Aa1, Cry26Aa1, Cry27Aa1, Cry28Aa1, Cry28Aa2, Cry29Aa1, Cry30Aa1, Cry30Ba1, Cry31Aa1, Cry31Aa2, Cry32Aa1, Cry32Ba1, Cry32Ca1, Cry32Da1, Cry33Aa1, Cry34Aa1, Cry34Aa2, Cry34Ab1, Cry34Ac1, Cry34Ac2, Cry34Ba1, Cry35Aa1, Cry35Aa2, Cry35Ab1, Cry35Ab2, Cry35Ac1, Cry35Ba1, Cry36Aa1, Cry37Aa1, Cry38Aa1, Cry39Aa1, Cry40Aa1, Cry40Ba1, Cry41Aa1, Cry41Ab1, Cry42Aa1, Cry43Aa1, Cry43Ba1, Cry44Aa, Cry45Aa, Cry46Aa, Cry47Aa, Cyt1Aa1, Cyt1Aa2, Cyt1Aa3, Cyt1Aa4, Cyt1Aa5, Cyt1Ab1, Cyt1Ba1, Cyt2Aa1, Cyt2Aa2, Cyt2Ba1, Cyt2Ba2, Cyt2Ba3, Cyt2Ba4, Cyt2Ba5, Cyt2Ba6, Cyt2Ba7, Cyt2Ba8, Cyt2Ba9, Cyt2Bb1, Cyt2Bc1, Cyt2Ca1, Vip3A(a) and Vip3A(b) and Vip3a(b).

8: The transgenic turf grass of claim 1, wherein said insect-resistant gene is obtained from a microorganism chosen from Serratia species, Photorhabdus species, and Xenorhabdus species.

9: The Serratia species of claim 8, wherein said species includes S. proteamaculans and S. entomophila.

10: The Photorhabdus species of claim 8, wherein said species includes P. fluorescens.

11: The Xenorhabdus species of claim 8, wherein said species includes X. nematophila and X. bovienii.

12: The transgenic turf grass of claim 1, wherein said insect-resistant gene comprises the cry8Ca gene from the Bacillus thuringiensis strain buibui.

13: The transgenic turf grass of claim 1, wherein said insect-resistant gene comprises the cry43A gene from Bacillus popilliae.

14: The transgenic turf grass of claim 1, wherein the insect-resistant gene comprises the Bacillus thuringiensis cry1Ca gene.

15: The transgenic turf grass of claim 1, comprising the polynucleotide sequence of SEQ ID NO:10

16: A method to reduce or eliminate any chemical or biological insecticide spray on turf grass to control insect pests by introducing one or more insect-resistant genes to turf grass.

17: The transgenic turf grass of claim 1, wherein the grass species is chosen from Alkali Sacaton (Sporobolus airoides); Altai Wildrye (Leymus angustus); Annual Ryegrass (Lolium multiflorum); Bahiagrass (Paspalum notatum); Barley (Elyhordeum); Bermudagrass (Cynodon dactylon); Bluestem (Andropogon); Bromegrass (Bromus); Broomcorn Millet (Panicum miliaceum); Browntop (Microstegium); Buckwheat (Eriogonum); Buffalograss (Buchloe dactyloides); Bulbous Canarygrass (Phalaris aquatica); California brome, Alaska brome (Bromus sitchensis); Canada Bluegrass (Poa compressa); Canarygrass (Phalaris); Chewings Fescue (Festuca rubra); Cockspur Grass (Echinochloa); Colonial Bentgrass (Agrostis tenuis); Common Barley (Hordeum vulgare); Common Wheat (Triticum aestivum); Creeping Bentgrass (Agrostis stolonifera); Creeping Meadow Foxtail (Alopecurus arundinaceus); Crested Wheatgrass (Agropyron cristatum); Dahurian Wildrye (Elymus dahuricus); Dallisgrass (Paspalum dilatatum); Fescue (Festuca); Fineleaf Sheep Fescue (Festuca filiformis); Finger Millet (Eleusine coracana); Gamagrass (Tripsacum); Grain Sorghum (Sorghum bicolor); Grama (Bouteloua); Grazing Bromegrass (Bromus stamineus); Hard Fescue (Festuca trachyphylla); Indiangrass (Sorghastrum nutans); Intermediate Wheatgrass (Thinopyrum intermedium); Japanese Millet (Echinochloa esculenta); Kentucky Bluegrass (Poa pratensis); Kikuyugrass (Pennisetum clandestinum); Klinegrass (Panicum coloratum); Lovegrass (Eragrostis); Meadow Brome (Bromus commutatus); Meadow Fescue (Festuca pratensis); Meadow Foxtail (Alopecurus pratensis); Meadow Ryegrass (Lolium pratense); Milletgrass (Milium); Oat (Avena); Orchardgrass (Dactylis glomerata); Pearlmillet (Pennisetum americanum); Perennial Ryegrass (Lolium perenne); Prairie grass (Bromus wildenowii); Prairie Junegrass (Koeleria macrantha); Rat-Tail Fescue (Vulpia myuros); Red Fescue (Festuca rubra); Redtop (Agrostis gigantea); Reed Canarygrass (Phalaris arundinacea); Rough Bluegrass (Poa trivialis); Rush Wheatgrass (Thinopyrum ponticum); Russian Wildrye (Psathyrostachys juncea); Rye (Secale cereale); Ryegrass (Lolium); Sheep Fescue (Festuca ovina); Slender Creeping Red Fescue (Festuca rubra); Smooth Bromegrass (Bromus inermis); Sorghum (Sorghum bicolor); Streambank Wheatgrass (Elymus lanceolatus); Sudangrass (Sorghum bicolor); Switchgrass (Panicum virgatum); Tall Fescue (Lolium arundinaceum); Tall Oatgrass (Arrhenatherum elatius); Tall Wheatgrass (Thinopyrum ponticum); Timothy (Phleum pratense); Triticale (Triticosecale rimpaui); Tufted Hairgrass (Deschampsia caespitosa); Western Wheatgrass (Pascopyrum smithii); Wheat (Triticum); Wheatgrass (Agropyron); and Wildrye (Elymus).

18: A method of creating insect-resistant turf grass, comprising:
a) introducing one or more plasmids comprising one or more insect-resistant genes into one or more turf grass calli wherein said calli are transformed;
b) culturing said calli;
c) growing said calli into mature turf grass; and
d) testing said mature turf grass for insect resistance.

19: The transgenic turf grass of claim 16, wherein the grass species is chosen from Alkali Sacaton (Sporobolus airoides); Altai Wildrye (Leymus angustus); Annual Ryegrass (Lolium multiflorum); Bahiagrass (Paspalum notatum); Barley (Elyhordeum); Bermudagrass (Cynodon dactylon); Bluestem (Andropogon); Bromegrass (Bromus); Broomcorn Millet (Panicum miliaceum); Browntop (Microstegium); Buckwheat (Eriogonum); Buffalograss (Buchloe dactyloides); Bulbous Canarygrass (Phalaris aquatica); California brome, Alaska brome (Bromus sitchensis); Canada Bluegrass (Poa compressa); Canarygrass (Phalaris); Chewings Fescue (Festuca rubra); Cockspur Grass (Echinochloa); Colonial Bentgrass (Agrostis tenuis); Common Barley (Hordeum vulgare); Common Wheat (Triticum aestivum); Creeping Bentgrass (Agrostis stolonifera); Creeping Meadow Foxtail (Alopecurus arundinaceus); Crested Wheatgrass (Agropyron cristatum); Dahurian Wildrye (Elymus dahuricus); Dallisgrass (Paspalum dilatatum); Fescue (Festuca); Fineleaf Sheep Fescue (Festuca filiformis); Finger Millet (Eleusine coracana); Gamagrass (Tripsacum); Grain Sorghum (Sorghum bicolor); Grama (Bouteloua); Grazing Bromegrass (Bromus stamineus); Hard Fescue (Festuca trachyphylla); Indiangrass (Sorghastrum nutans); Intermediate Wheatgrass (Thinopyrum intermedium); Japanese Millet (Echinochloa esculenta); Kentucky Bluegrass (Poa pratensis); Kikuyugrass (Pennisetum clandestinum); Klinegrass (Panicum coloratum); Lovegrass (Eragrostis); Meadow Brome (Bromus commutatus); Meadow Fescue (Festuca pratensis); Meadow Foxtail (Alopecurus pratensis); Meadow Ryegrass (Lolium pratense); Milletgrass (Milium); Oat (Avena); Orchardgrass (Dactylis glomerata); Pearlmillet (Pennisetum americanum); Perennial Ryegrass (Lolium perenne); Prairie grass (Bromus wildenowii); Prairie Junegrass (Koeleria macrantha); Rat-Tail Fescue (Vulpia myuros); Red Fescue (Festuca rubra); Redtop (Agrostis gigantea); Reed Canarygrass (Phalaris arundinacea); Rough Bluegrass (Poa trivialis); Rush Wheatgrass (Thinopyrum ponticum); Russian Wildrye (Psathyrostachys juncea); Rye (Secale cereale); Ryegrass (Lolium); Sheep Fescue (Festuca ovina); Slender Creeping Red Fescue (Festuca rubra); Smooth Bromegrass (Bromus inermis); Sorghum (Sorghum bicolor); Streambank Wheatgrass (Elymus lanceolatus); Sudangrass (Sorghum bicolor); Switchgrass (Panicum virgatum); Tall Fescue (Lolium arundinaceum); Tall Oatgrass (Arrhenatherum elatius); Tall Wheatgrass (Thinopyrum ponticum); Timothy (Phleum pratense); Triticale (Triticosecale rimpaui); Tufted Hairgrass (Deschampsia caespitosa); Western Wheatgrass (Pascopyrum smithii); Wheat (Triticum); Wheatgrass (Agropyron); and Wildrye (Elymus).


SEQUENCE LISTING (14 sequences)

SEQ ID NO: 1 - DNA, 29 bp, Artificial Sequence
  Synthetic Construct
ggatcccatgagtccaaataatcaaaatg

SEQ ID NO: 2 - DNA, 30 bp, Artificial Sequence
  Synthetic Construct
cccgggtcacacatctaggtcttcttctgc

SEQ ID NO: 3 - DNA, 5869 bp, Artificial Sequence
  Plasmid pB1221-8D1
aagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagaggatcccatgagtccaaataatcaaaatgaatatgaaattctagatgcttcatcatctacttctgtatccgataattctgttagataccctttagcaaacgatcaaacgaccacattacaaaacatgaactataaagattatctgagaatgtctgagggagagaatcctgaattatttggaaatccggagacgtttattagttcatctacggttcaaactggaattggcattgttggtcaagtactgggggctttaggggttccatttgctggacagatagctagtttttatagtttcattgtcggtcaattatggccatcaagtaccgtgagtgtatgggaaatgattatgaaacaagtggaagatctaattgatcaaaaaataacagattctgtaaggaaaacagcgcttgcaggactacaaggattaggagatggcttagacgtatatcagaaatcacttaagaattggctggaaaatcgtaatgatacaagagctagaagtgttgtggtgacccaatatatagctttagagcttgattttgttgctaaaatcccatcttttgcaatatctggacaggaagtaccattattatcagtgtatgcacaagcagcgaatttacatttgctattattacgagatgcttccatttttggagcagagtggggattcacaccaggagaaatttccacattttatgatcgtcaggtgacacgtaccgcccaatactcggattattgtgtaaagtggtataacactggcttagataaattaaaaggtacgaatgctgcaagttggctgaagtatcaccaattccgaagagaaatgacattactggtattagatttagtagcgttatttccaaactatgacacacgtacgtatccaatcgaaacaacggcccaacttacacgggaagtgtatacagatccaatagtatttaacagagaaacaagtggtggattttgtaggcgttggtcacttaacagtgatatttctttttcagaagtcgaaagcgctgtaattcgttcaccacacctatttgatatactcagtgaaatagaattttatacaacaagagcggggcttcccttgaataatacggaataccttgaatattgggtaggacattctataaaatataaaaatacgaatgcctcatcagcattagaacgtaattacggtacgattacttctaacaaaatcaagtattatgatttagcaaataaggatatctttcaggttcgatcattaggggcggatttagctaattactacgcacaggtatatggagttccgtacgctagttttacactgcttgacaagaatacaggatcaggatcagttggaggttttacgtactcaaaaccacatacaactatgcaagtatgtacacaaaattacaatacgattgatgaaatccctccagagaatgagccacttagtagagggtatagccatagattatctcatatcacctcttattctttttctaagaatgctagtagtcctgctagatatggcaatctccctgtatttgcttggacacatcggagtgcggatgttacaaatacagtttattcagataaaattactcagataccagttgtaaaggcacatactttagtttcaggtactactgttattaaaggtcctggatttacaggaggcaatatccttaaaagaacaagtagtggtccgttagcttatactagtgtctctgtaaaatcaccattatcacaaagatatcgtgcaagaatacgttatgcttctactactaacttacgactttttgtaacaatttctggaactcgcatttactctataaatgttaataaaaccatgaataaaggggatgatttaacatttaatacatttgacttagcaactattggtactgctttcacattttcaaattactcggatagcttaacggtaggtgcagattcttttgcttcaggaggagaagtttatgtagataagttcgaacttattccggtaaatgcaacatttgaagcagaagaagacctagatgtgtgacccgggaatcactagtgaattcgcggccgcctgcaggtcgaccatatgggagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 4 - DNA, 31 bp, Artificial Sequence
  Synthetic Construct
ggatccatgaatcagtatcataaccaaaacg

SEQ ID NO: 5 - DNA, 33 bp, Artificial Sequence
  Synthetic Construct
cccgggttacttttccatacaaatcaattccac

SEQ ID NO: 6 - DNA, 30 bp, Artificial Sequence
  Synthetic Construct
ggatccatgagtccaaataatcaaaatgag

SEQ ID NO: 7 - DNA, 33 bp, Artificial Sequence
  Synthetic Construct
cccgggttactcttcttctaacacgagttctac

SEQ ID NO: 8 - DNA, 34 bp, Artificial Sequence
  Synthetic Construct
ggatcccatggaggaaaataatcaaaatcaatgc

SEQ ID NO: 9 - DNA, 31 bp, Artificial Sequence
  Synthetic Construct
cccgggttattcctccataaggagtaattcc

SEQ ID NO: 10 - DNA, 3460 bp, Bacillus thuringiensis strain SDS-502
ggatccccatggcaagcccgaacaatcagaacgagtatgagattctggacgcatccagctcaacctcagtgtctgataactccgtgcggtaccctttggcgaacgaccagactaccactctccagaacatgaactataaggattacctgaggatgtctgagggagaaaatccggagttgttcgggaatcctgagacgtttatttcctcctcaacggtccaaactgggatcggaattgttggtcaggtcttgggtgcgcttggtgtcccgttcgcaggtcagatcgcttccttctattcattcatcgtcggacagctgtggccgtctagtaccgtgagcgtgtgggagatgattatgaagcaagtcgaagacttgattgaccaaaagatcactgattcagttcgtaagactgctcttgctgggttgcaagggttaggtgatggactcgacgtttatcagaaaagtctcaagaattggttggaaaaccgaaacgatacacgagctcgctccgtcgtggttactcaatacatcgcactggaacttgacttcgttgcgaagatccctagcttcgcgatctccggtcaagaggtccccttgctctcggtttacgctcaggcagctaacctgcatttgttgttactgcgtgatgcaagtatctttggagctgaatggggttttacccctggggaaatctccacattctacgacagacaggttacaaggacggcgcagtactccgactattgtgtgaagtggtacaatacaggactcgataagttgaagggcactaacgcagccagctggttaaaataccaccagttccgtcgggagatgactttgctcgtgcttgacctcgttgccctcttccctaactatgacactcgaacctacccaatcgagacgacagcgcagctgactcgcgaggtttatactgaccctatcgtcttcaacagagagaccagcggcgggttttgccggcgatggtccttgaactcggacatctccttcagtgaagtcgagtcggccgtcatccgttccccccatctcttcgacatattgtccgaaatcgagttctatacgacccgggcgggcttgccactgaacaatactgagtacttggagtactgggtggggcacagtattaagtataagaataccaacgcgtcctctgcgctggaacgaaactatgggactatcacgtccaacaaaattaagtattacgacctcgctaacaaagatatcttccaagtgagatccctgggagccgacctcgcgaactactacgcccaagtctacggtgtcccatatgcgagcttcaccctgctcgacaagaatacgggctctgggtccgtcggcgggtttacttactcaaaaccgcataccactatgcaggtgtgtacacagaattacaacaccatcgatgagatcccacctgaaaacgagcctctcagtaggggatactcccatcggttgagtcacatcacgtcctactcttttagcaagaacgcatcgtcccctgcgcgatatgggaatctgccggttttcgcttggactcaccgctccgccgacgttactaacaccgtttactccgacaaaatcacacagatcccggtcgtcaaagcccacacgctggtgtccggtaccaccgtgatcaagggcccaggcttcaccggtggcaacatccttaaacgtacttctagtggtcctctggcttatacctctgtgtcggtaaaatcgcctctctcccaacgctacagggcccggattcgctacgctagcaccactaacttgcgcttgttcgtgaccatctccggtacacgcatctactcaattaacgtcaacaagaccatgaacaagggagacgatttgaccttcaatacgttcgatcttgctaccatcggaaccgcatttaccttttcgaattactccgactccttaacagtgggcgcagactcgttcgccagcggcggtgaagtttacgttgacaaattcgaactcattcccgtcaatgccacattcgaagctgaagaggatttggacgttgccaagaaggcagtgaagaatctggtcgaatgcctgtctgacgagttgtatccgaacgagaaaagaatgctgtgggacgccgtcaaagaagcaaagcggcttgtgcaggcgcggaatctcttgcaagatacgggctttaaccgaatcaacggagaaaacggctggaccggatcgacagggattgaggtggcagagggtgacgtcttgttcaaggatcgctccttgcgactaacttccgcacgggagattgatactgaaacctaccccacttacttgtatcagcaaatcgacgaaagtttgcttaaaccttacaccaggtataaattgaaaggattcatcgggtcctctcaagatcttgagatcaagttgattagacaccgggcgaatcaaattgtcaagaatgtccccgacaatttgttgcctgacgttctcccggtcaactcttgtggtggaatcgacaggtgttctgagcaacagtatgtggatgcgaatttggcactcgagaataacggcgagaacggaaacatgtcctccgactcccatgccttctccttccacatcgacaccggagagatcgacctcaacgaaaacaccggtatttgggtggtgttcaagattcccaccacaaatggttatgcaacgctcggcaatctcgaactcgtagaggaaggcccgctgtcgggagaaactctggaacgcgcacaacagcaagagcaacaatggcaggacaagatggctagaaaacggggcgcttctgaaaaggcctactacgccgcaaaacaagccatcgaccggctgtttgctgactatcaggaccagaaactcaattccggtgtcgaaatgagtgatatgttggccgcccaaaatttggtccagtccattccttacgtgtacaacgatgccttgcccgagatccctggcatgaactacacttcgtttacggagctcacaaatcgcctccagcaggcatggaatctgtatgatcttcggaacgccattccaaacggcgattttcggaacggcctgagcgattggaacgccaccagcgatgtaaacgtccagcagttgtccgatacatccgtcctcgtcatccccaattggaacagccaagttagccaacagtttaccgttcaaccgaactaccggtacgtgcttcgcgtcaccgcgaggaaagaaggagtcggcgacggctatgtcattatcagggacggggcgaaccaaacagagacattgacctttaacatttgcgacgacgacaccggggtcttgtcggctgaccaaacttcctatatcacgaagactgtggagttcactccttccacggagcaggtgtggatcgatatgtcagagaccgaaggtgtatttaatatcgagtccgtcgagttggtgctagaagaggaatagtctagagcggccgc

SEQ ID NO: 11 - DNA, 31 bp, Artificial Sequence
  Synthetic Construct
ccggaattcgatcatgagcggagaattaagg

SEQ ID NO: 12 - DNA, 34 bp, Artificial Sequence
  Synthetic Construct
ccggaattcatcttgaaagaaatatagtttaaat

SEQ ID NO: 13 - DNA, 29 bp, Artificial Sequence
  Synthetic Construct
ggatcccatgagtccaaataatcaaaatg

SEQ ID NO: 14 - DNA, 30 bp, Artificial Sequence; identical to SEQ ID NO: 2
  Synthetic Construct